(19) United States
(12) Patent Application Publication
(10) Pub. No.: US 2022/0362226 A1
(43) Pub. Date: Nov. 17, 2022

(54) COMPOSITIONS AND METHODS FOR TREATING PRURITUS

(71) Applicants: LUMOSA THERAPEUTICS CO., LTD., Taipei (TW); SHANGHAI LUMOSA THERAPEUTICS., LTD., Shanghai (CN)

(72) Inventors: Wendy Huang CHERN, Taipei City (TW); Chan-Jung LI, Taipei City (TW); Shu-Wen KUO, Taipei City (TW); David Chih-Kuang CHOU, Taipei City (TW); Yu-En TIEN, Taipei City (TW)

(21) Appl. No.: 17/661,444

(22) Filed: Apr. 29, 2022

Related U.S. Application Data
(63) Continuation of application No. 16/368,609, filed on Mar. 28, 2019, now abandoned.
(60) Provisional application No. 62/650,108, filed on Mar. 29, 2018.

(51) Int. Cl. A61K 31/439; A61P 17/04; A61K 9/00; A61K 47/34; A61K 47/14; A61K 47/12; A61K 47/38; A61K 47/44; A61K 47/26

(52) U.S. Cl. CPC A61K 31/439; A61P 17/04; A61K 9/0014; A61K 47/34; A61K 47/14; A61K 47/12; A61K 47/38; A61K 47/44; A61K 47/26

(57) ABSTRACT

Disclosed herein are topical pharmaceutical compositions of Sebacoyl Dinalbuphine Ester (SDE) and methods of using such compositions for treating pruritus, pain, and inflammatory conditions.

### CROSS-REFERENCE TO RELATED APPLICATIONS

This application claims priority to U.S. Application No. 62/650,108 filed Mar. 29, 2018, which is incorporated herein by reference in its entirety.

### FIELD

The present invention relates to topical pharmaceutical compositions, formulated with Sebacoyl Dinalbuphine Ester (SDE, also called dinalbuphine sebacate), and methods of treating pruritus and related conditions, as well as pain and/or inflammation, through topical administration of the compositions disclosed herein.

### BACKGROUND AND SUMMARY

Skin is the largest organ of the human body. It is a heterogeneous multilayer tissue, and its primary function is to protect the body from the external environment by functioning as an effective barrier to absorption of exogenous molecules. In general, human skin includes two layers, an outer layer, the epidermis, and an underlying layer, the dermis. The dermis is a connective layer that is responsible for the elasticity of skin and is composed of three major types of cells, fibroblasts, macrophages, and adipocytes. Additionally, the dermis is composed of a matrix of components such as collagen, elastin, and extrafibrillar matrix. The epidermis primarily functions to regulate secretion and body temperature, and protect against dehydration and infection. Skin ailments or disorders, however, tend to impair the functions of the epidermis, and may limit the outer layer's ability to protect the body. Indeed, skin conditions and dermal irritations are common problems for many individuals.

The dermis thickness ranges from 3 to 5 mm and contains a mixture of fibrous proteins (collagen and elastin) and an interfibrillar gel of glycosaminoglycans, salts, and water. Collagen types I and II account for approximately 75% of the dry weight of the dermis. Blood and lymph vessels, free nerve endings, hair follicles, and sebaceous and sweat glands are embedded in the dermis. The hair follicles and sweat gland ducts open directly to the outside on the surface of the skin.

The epidermis, excluding the stratum corneum, which is the outermost layer, is a viable tissue. The epidermis is not vascularized, and nutrients diffuse from the dermoepidermal junction to maintain its viability. There are five layers that represent the different stages of cell life in the epidermis. The sequence of layers from inside to outside are the germinative (or basal) layer, stratum spinosum, stratum granulosum, stratum lucidum, and stratum corneum. The stratum corneum cells, corneocytes, are dense, functionally dead, anucleated, and filled with keratin. The stratum corneum arrangement is densely packed with corneocytes and intercellular lipids which form several bilayers surrounding the corneocytes.

Pruritus, or itch, is an uncomfortable skin sensation that provokes a desire to scratch. It may involve the entire skin (generalized pruritus) or only particular areas, such as the scalp, upper back, arms, or groin (localized pruritus). Although itch may be acute, for example, from a bug bite, chronic pruritus originates from many different causes. It is a debilitating condition, comparable to chronic pain, which negatively impacts quality of life. For example, pruritus can cause anger, a feeling of helplessness, and frustration, and the relentless itch can significantly disrupt sleep and concentration. See Blume-Peytavi et al., Atopic dermatitis in children: management of pruritus, J. Eur. Acad. Dermatol. Venereol., 26:2-8, 2012; Chang et al., Atopic dermatitis, melatonin, and sleep disturbance, Pediatrics, 134:e397-405, 2014.

Chronic pruritus affects millions of people worldwide, although solid epidemiological data are very limited. Patients with certain diseases and conditions report high incidences of chronic itch, including those with psoriasis, Hodgkin's disease, dialysis patients, and polycythaemica vera. See Metz et al., CME Dermatol., 3:3, 124-143, 2008. Chronic pruritus is also a prevalent symptom in cutaneous T-cell lymphoma, a disease that includes mycosis fungoids and Sezary syndrome. See Meyer et al., Acta Derm, Venereol., 90:12-17, 2010. Pruritus is the most common dermatological complaint in elderly patients. See Beauregard et al., Arch. Dermatol., 123:1638-43, 1987. Itch is often a side effect of certain drugs, such as EGF receptor antagonists. Hu et al., Cutaneous side effects of epidermal growth factor receptor inhibitors: clinical presentation, pathogenesis, and management, J. Am. Acad. Dermatol., 55(2):317-26, 2007.

Antihistamines can sometimes effectively treat itch due to acute urticaria, but many chronic pruritic diseases respond poorly to conventional H1 receptor antagonists. Tey et al., Br. J. Dermatol., 165(1):5-7, 2011. In addition to marginal efficacy, antihistamines can also cause intolerable drowsiness. Other current therapies possess various limitations. For example, anticonvulsants such a gabapentin inhibit spinal mechanisms in the perception of itch, but their use is limited due to their slow onset of action. Opiate receptor antagonists such a naloxone, nalmefene, and naltrexone decreased pruritus symptoms in patients with liver and kidney disease, although significant central nervous and gastrointestinal side effects occurred. Bergasa et al., Hepatology, 44(5):1317-23, 2006.

Topical corticosteroids are a first line therapy for acute pruritus associated with inflammatory skin diseases. While the exact mechanism of action is not known, topical corticosteroids are thought to activate glucocorticoid receptors that inhibit cytokine activation, thereby decreasing local inflammation and indirectly controlling pruritus. Thus, while frequently employed by health practitioners to treat patients with pruritus of unknown etiology, it must be emphasized that topical corticosteroids are of limited to no benefit in patients with non-inflammatory itch. See Elmariah et al., Topical Therapies for Pruritus, Semin. Cutan. Med. Surg. 30(2):118-126, 2011.

Skin permeation is a significant obstacle to developing effective topical medicaments targeting pruritus. Permeation routes include transport across the epidermis and skin appendages, particularly the hair follicles and sweat glands that form an alternative pathway to the intact epidermis. The skin appendages represent only 0.1% of the total surface area of the human skin, and the contribution of this route for permeation flux of drugs is small. Recently, it was proposed that the route through skin appendages contributes little to the rate of skin absorption of most drugs in the steady state; however, this route enables permeation of charged molecules and large polar compounds. The major route of skin permeation is through the intact epidermis, and two main pathways have been identified: the intracellular route though the lipids of the stratum corneum and the transcellular route through the corneocytes. In both cases, the drug must diffuse into the intercellular lipid matrix, which is recognized as the major determinant of drug absorption by the skin. See Alexander et al., Approaches for breaking the barriers of drug permeation through transdermal drug delivery, J. Control. Rev., 164(1):26-40, 2012; Desai et al., Investigation of follicular and non-follicular pathways for polyarginine and oleic acid modified nanoparticles, Pharm. Res., 30(4):1037-49, 2013. Diffusion through the intercellular lipid matrix is thought to be limited to smaller molecules with a molecular weight below 500 Da. Naik, et al. Transdermal drug delivery: overcoming the skin's barrier function. Pharm Sci Technol Today, 3:318-26, 2000. This is problematic for larger drugs, which represent the majority of active agents for therapeutic applications. Bos et al., The 500 Dalton rule for skin penetration of chemical compounds and drugs, Exp. Dermatol., 9(3):165-9, 2000.

Opiates are drugs derived from opium and include morphine, codeine, and a wide variety of semisynthetic opioid congeners. Opioids include the opiates and all agonists and antagonists with morphine-like activity and naturally occurring endogenous and synthetic opioid peptides. Although morphine and other morphine-like opioid agonists are commonly used to produce analgesia, the severity and high incidence of side effects limits their use. Common side effects of opioid administration include sedation, dizziness, nausea, vomiting, constipation, and respiratory depression. Physical dependence, tolerance, and addiction are also clinical concerns. The most common side effects of opioid usage are constipation and nausea, which can be difficult to manage and tolerance frequently does not develop. See Benyamin et al., Opioid complications and side effects, Pain Physician, 11(2 Suppl):5105-20, 2008.

There are three classical types of opioid receptors that have been investigated as the mediators of opiate effects. These opioid receptors are classified as mu (“μ”), kappa (“κ”), and delta (“δ”). Nalbuphine is a derivative of 14-hydroxymorphine and is structurally related to the opioid μ-receptor agonist oxymorphone and the opioid μ-receptor antagonist, naloxone. Gustein et al. (Chapter 23: Opioid Analgesics, Goodman & Gilman's The Pharmacologic Basis of Therapeutics, 10^{th} Ed., McGraw Hill 2001, pp. 569-619) report that nalbuphine exerts its clinical pharmacologic action by competitively antagonizing the opioid μ-receptor and simultaneously acting as an agonist at the opioid κ-receptor, and thus is a member of the “opioid agonist-antagonist” class of drugs that mechanistically work though this dual pharmacologic process. Nalbuphine has been used to treat acute, chronic, and post-surgical pain.

Sebacoyl Dinalbuphine Ester (SDE), also called dinalbuphine sebacate, is a prodrug of nalbuphine with a molecular weight of 881 Da. An advantage of prodrugs, particularly SDE, is their long-term efficacy and controlled release, enhancing the effective time of a single dose. The SDE molecule includes two nalbuphine molecules esterified through a sebacic acid linker. Ester linkages of prodrugs tend to be hydrolyzed efficiently because of the wide availability of endogenous esterases, allowing for continuous release of active drug.

Sung and coworkers have reported that nalbuphine and certain low molecular weight nalbuphine prodrugs with increased lipophilicity (nalbuphine pivalate, nalbuphine enanthate, and nalbuphine dacanoate) can diffuse through skin. See Sung et al., Delivery of nalbuphine and its prodrugs across skin by passive diffusion and iontophoresis, Journal of Controlled Release, 67:1-8, 2000. In contrast, these workers subsequently reported that SDE “exceeded the cut-off point for passive permeation through the skin” and, therefore, was not appropriate for transdermal delivery. Huang et al., Int. J. Pharmaceu., 297:162-171, 2005. In line with these findings, formulations of nalbuphine and other low molecular weight opiate analogs, but not of SDE, have been proposed as treatments for pruritus. See U.S. Pat. No. 9,624,233; U.S. Pub. No. 2014/0179727. However, systemic administration of nalbuphine at effective doses for treating pruritus lead to adverse effects, such as nausea, vomiting and somnolence. (See Table 4 of Am J Nephrol 2017; 46:450-458) The embodiments disclosed herein are based on the surprising finding that SDE in certain formulations is capable of diffusing into the skin and, despite earlier reports to the contrary, is suitable for topical administration.

In various embodiments, a non-aqueous pharmaceutical composition for topical use comprises sebacoyl dinalbuphine ester (SDE) or a pharmaceutically acceptable salt thereof and at least one pharmaceutically acceptable excipient.

In various embodiments, a method of treating pruritus conditions, pain, and/or inflammatory conditions comprises administering a pharmaceutically effective amount of a pharmaceutical composition disclosed herein to a subject in need thereof.

In some embodiments, the invention comprises a non-aqueous pharmaceutical composition for topical use comprising sebacoyl dinalbuphine ester (SDE) or a pharmaceutically acceptable salt thereof and at least one pharmaceutically acceptable excipient.

In various embodiments, at least one pharmaceutically acceptable excipient is a penetration enhancer. In various embodiments, the penetration enhancer is selected from dimethyl isosorbide, diethylene glycol monoethyl ether, castor oil and oleyl alcohol.

In some embodiments, at least one pharmaceutically acceptable excipient is a thickening agent. In various embodiments, the thickening agent is selected from PEG 4000, soft paraffin, hydroxypropyl cellulose (HPC), and stearic acid

In some embodiments, at least one pharmaceutically acceptable excipient is a solvent. In certain embodiments, the solvent is selected from PEG 400, diisopropyl adipate, benzyl benzoate, N-methyl-2-pyrrolidone, isopropyl myristate (IPM), caprylic/capric triglyceride, and liquid paraffin.

In various embodiments, at least one pharmaceutically acceptable excipient is an antioxidant. In some embodiments, the antioxidant is selected from butylated hydroxytoluene (BHT), butylated hydroxyanisole (BHA), ethylenediaminetetraacetic acid (EDTA), propyl gallate, ascorbic acid, citric acid, ascorbyl palmitate, alpha-tocopherol, and alpha-tocopherol acetate.

Certain pharmaceutical compositions of the disclosure comprise a solvent, a thickening agent, and a penetration enhancer.

Particular pharmaceutical compositions of the disclosure comprise from about 0.1 to about 5 percent (w/w) SDE, from about 34 to about 85 percent (w/w) solvent, from about 0.8 to about 40 percent (w/w) thickening agent, and from about 0 to 65 percent (w/w) penetration enhancer.

Other pharmaceutical compositions of the disclosure comprise from about 0.1 to about 2 percent (w/w) SDE, from about 0 to about 15 percent (w/w) dimethyl isosorbide, from about 44 to about 70 percent (w/w) PEG 400, from about 0 to 10 percent (w/w) diethylene glycol monoethyl ether, about 15 percent (w/w) diisopropyl adipate, and about 16% (w/w) PEG 4000.

Various pharmaceutical compositions of the disclosure comprise from about 0.1 to about 5.1 percent (w/w) SDE, from about 0 to about 15 percent (w/w) dimethyl isosorbide, from about 14 to about 65 percent (w/w) PEG 400, from about 10 to about 50 percent (w/w) diethylene glycol monoethyl ether, from about 15 to about 20 percent (w/w) diisopropyl adipate, about 0.8 percent (w/w) hydroxypropylcellulose.

Certain pharmaceutical composition of the disclosure comprise about 0.1-4 percent (w/w) SDE, about 15 percent (w/w) dimethyl isosorbide, 14.1 percent (w/w) PEG 400, about 50 percent (w/w) diethylene glycol monoethyl ether, about 20 percent (w/w) diisopropyl adipate, and about 0.8 percent (w/w) hydroxypropylcellulose.

Some of the pharmaceutical compositions of the disclosure comprise about 4 percent (w/w) SDE.

Various pharmaceutical compositions of the disclosure comprise from about 0.1 to about 3.2 percent (w/w) SDE, from about 5 to about 25 percent (w/w) isopropyl myristate, from about 0 to about 10 percent (w/w) oleyl alcohol, from about 0 to about 20 percent (w/w) castor oil, about 5 to about 15 percent (w/w) Caprylic/Capric Triglyceride, about 0 to about 25 percent (w/w) liquid paraffin, about 10 to about 30 percent (w/w) diisopropyl adipate, about 26 to about 45 percent (w/w) soft paraffin, and about 0 to 8 percent (w/w) stearic acid.

Some of the pharmaceutical compositions of the disclosure comprise from about 0.1 to about 2 percent (w/w) SDE, about 16 percent (w/w) isopropyl myristate, from about 14 percent (w/w) castor oil, about 10 percent (w/w) Caprylic/Capric Triglyceride, about 20 percent (w/w) diisopropyl adipate, about 32 percent (w/w) soft paraffin, and about 0-8 percent (w/w) stearic acid.

In certain embodiments, the disclosure provides a method of treating disorders, comprising topically administering a pharmaceutically effective amount of a pharmaceutical composition comprising SDE or a pharmaceutically acceptable salt thereof and at least one pharmaceutically acceptable excipient to a subject in need thereof.

In some embodiments, the disclosure provides a method of treating disorders selected from pruritus, pain, or inflammatory conditions.

In various embodiments, the pruritus condition is uremic pruritus, atopic dermatitis, or prurigo nodularis.

In some embodiments of the disclosed method, the pharmaceutical composition is administered three times a day, twice a day, once a day, or every 2, 3, 4, 5, 6, or 7 days.

In some embodiments of the disclosed method, the pharmaceutical composition releases nalbuphine from SDE over the course of 2, 4, 6, 8, 12, 24, 48, or 72 hours.

In certain embodiments of the disclosed method, the pharmaceutical composition is administered as a topical gel, topical ointment, topical lotion, topical form, or topical cream.

In some embodiments of the disclosed method, the SDE concentration in the dermis and epidermis at 24 hours post administration is higher than the SDE concentration in the circulation.

In various embodiments of the disclosed method, the symptoms of pruritus are alleviated or partially alleviated.

In some embodiments of the disclosed method, the subject does not experience opioid-related side effects.

### BRIEF DESCRIPTION OF THE DRAWINGS

FIG. 1A. SDE accumulation in the epidermis, dermis, and receiver fluid.

FIG. 1B. Nalbuphine accumulation in the epidermis, dermis, and receiver fluid.

FIG. 2A. Comparison of SDE and nalbuphine accumulation in the epidermis.

FIG. 2B. Comparison of SDE and nalbuphine accumulation in the dermis.

FIG. 2C. Comparison of SDE and nalbuphine accumulation in the receiver fluid.

FIG. 3. Anti-pruritus effect of subcutaneous administration of SDE.

FIG. 4. Anti-pruritus effect of topical administration of SDE—non-aqueous gel formulation.

FIG. 5. Blood concentration of nalbuphine after topical administration of SDE—non-aqueous gel formulation.

FIG. 6A. SDE accumulation in the epidermis, dermis, and receiver fluid.

FIG. 6B. Nalbuphine accumulation in the epidermis, dermis, and receiver fluid.

FIG. 7. Anti-pruritus effect of topical administration of SDE—Non-aqueous ointment formulation.

FIG. 8. Blood concentration of nalbuphine after topical administration of SDE—non-aqueous gel formulation.

FIGS. 9A-B. Appearance of formulations P1-P8 after 2 weeks at 4 and 25° C. FIG. 9A shows formulations P1-P8 after 2 weeks at 25° C. FIG. 9B shows formulations P1-P8 after 2 weeks at 4° C.

### DETAILED DESCRIPTION

As used herein, the singular forms “a” “an” and “the” are intended to include the plural forms as well, unless the context clearly indicates otherwise. Furthermore, to the extent that the terms “having,” “has,” “or variants thereof are used in either the detailed description and/or the claims, such terms are intended to be inclusive in a manner similar to the term “comprising.”

As used herein, the terms “about” and “approximately” mean within an acceptable error range for the particular value as determined by one of ordinary skill in the art, which will depend in part on how the value is measured or determined, i.e., the limitations of the measurement system. In some embodiments, “about” means a range of plus or minus 10% of the stated value. Where particular values are described in the application and claims, unless otherwise stated the term “about” meaning within an acceptable error range for the particular value should be assumed. Also, all ranges described herein include the endpoints as well as all points in between. The term “or” will be understood to mean “and/or” unless the context clearly indicates otherwise.

The term “pharmaceutically acceptable” means biologically or pharmacologically compatible for in vivo use in animals or humans. In some embodiments, “pharmaceutically acceptable” means approved by a regulatory agency of the Federal or a state government or listed in the U.S. Pharmacopeia or other generally recognized pharmacopeia for use in animals, and more particularly in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional pharmaceutically acceptable carrier, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases. Accordingly, in some embodiments, the pharmaceutical compositions of the present invention encompass any composition made by admixing 0.1 to 75%, of the active ingredient with a pharmaceutically acceptable carrier. In other embodiments, the pharmaceutical compositions of the present invention encompass any composition made by admixing 0.1 to 50%, of the active ingredient with a pharmaceutically acceptable carrier. In yet other embodiments, the pharmaceutical compositions of the present invention encompass any composition made by admixing 0.1 to 25%, of the active ingredient with a pharmaceutically acceptable carrier. In certain embodiments, the pharmaceutical compositions of the present invention encompass any composition made by admixing 0.1 to 10%, of the active ingredient with a pharmaceutically acceptable carrier. In other embodiments, the pharmaceutical compositions of the present invention encompass any composition made by admixing 0.1 to 5%, of the active ingredient with a pharmaceutically acceptable carrier.

The term “non-aqueous” as used herein is intended to refer to formulations having a water content about 10% by weight or less. The term “non-aqueous” does not exclude trace amounts of residual water that come from any one or more of the components in the formulation. In some embodiments, non-aqueous formulations comprise less than about 5% water by weight, or less than about 3% water by weight, or less than about 2% water by weight, or less than about 1% water by weight, or less than about 0.5% water by weight.

The term “solvent” means a substance, usually a liquid, in which a solute dissolves to form a solution. As used herein, solvents include skin conditioner or an emollient that reduces evaporation and thus increases the moisture content of the skin. Examples of solvents include, but are not limited to, polyethylene glycol (PEG) 400, diisopropyl adipate, benzyl benzoate, N-methyl-2-pyrrolidone, sopropyl myristate (IPM), caprylic/capric triglyceride (medium chain triglycerides), and liquid paraffin (mineral oil) among others.

The term “penetration enhancer” means an agent that enhances penetration of drugs into the skin. Such agents perturb the skin barrier via extraction or fluidization of lipid bilayers aiding other molecules in crossing the skin barrier. Examples include, but are not limited to, dimethyl isosorbide, diethylene glycol monoethyl ether, castor oil, and oleyl alcohol among others.

The term “thickening agent” means a substance that can increase the viscosity of a liquid without substantially changing its other properties or substances specifically designed to make the epidermis softer and more pliable. Thickening agents may also improve the suspension of other ingredients or emulsions which increases the stability of the mixture. Examples include, but are not limited to, polyethylene glycol (PEG) 4000, soft paraffin (petrolatum), hydroxypropyl cellulose (HPC), and stearic acid among others.

The term “antioxidant” means a pharmaceutically acceptable excipient that stabilizes the formulation or provides protection against degradation such as oxidation. Examples include, but are not limited to, butylated hydroxytoluene (BHT), butylated hydroxyanisole (BHA), ethylenediaminetetraacetic acid (EDTA), propyl gallate, ascorbic acid, citric acid, ascorbyl palm itate, alpha-tocopherol and alpha-tocopherol acetate among others.

As used in this disclosure, the term “subject” includes, without limitation, a human or an animal. Exemplary animals include, but are not limited to, mammals such as mouse, rat, guinea pig, dog, cat, horse, cow, pig, monkey, chimpanzee, baboon, or rhesus monkey.

The term “treatment” as used herein, is defined as the application or administration of a therapeutic agent to a subject, who has pruritus, pain, inflammation, or a related condition, a symptom of pruritus, pain, inflammation, or is predisposed toward pruritus, pain, or inflammation, or a related condition, with the purpose to cure, heal, alleviate, relieve, alter, remedy, ameliorate, improve, or affect the condition, one or more symptoms of the condition, either alone or in combination with another therapeutic agent, as compared to that symptom in the absence of treatment. The result should be considered a treatment of the underlying disorder regardless of whether all or only some of the symptoms of the disorder are cured, healed, alleviated, relieved, altered, remedied, ameliorated, improved, or affected.

The term “pruritus” as used herein, is defined as a sensation driving the urge to scratch, may be acute or chronic. In various embodiments, the pruritus condition can be uremic pruritus, atopic dermatitis, prurigo, nervous dermatitis, aquagenic pruritus, atopic dermatitis, photosensitive dermatosis, idiopathic pruritus, Lichen simplex chronicus, prurigo nodularis, psoriasis, cholestatic pruritus, contact dermatitis, seborrheic dermatitis, autosensitization dermatitis, caterpillar dermatitis, eczema, asteatosis, senile pruritus cutaneous, insect sting, scabies, urticaria, herpes, impetigo, tinea, lichen, acne vulgaris, or visceral diseases complicated with pruritus, brachioradial pruritus, burn-induced pruritus, cancer-induced pruritus, neuropathic pruritus, morphine-induced pruritus, multiple sclerosis-associated pruritus, post-herpetic pruritus, bullous pemphigoid, Netherton syndrome, or itching from poison or any other stimulus,

As used herein, “SDE” means sebacoyl dinalbuphine ester (also called dinalbuphine sebacate) and/or pharmaceutically acceptable salts thereof.

### Compositions

Described herein are topical pharmaceutical compositions useful for treating pruritus, pain, and inflammatory condition. The compositions and methods are based on the surprising discovery that certain formulations of SDE are capable of penetrating the dermis and epidermis. SDE is larger than molecules typically thought to have epidermal and dermal penetrating properties. For instance, to allow for skin penetration, a pharmaceutical compound typically must have a molecular weight below 500 Da. See Naik et al., Transdermal drug delivery: overcoming the skin's barrier function, Pharm Sci Technol Today, 3:318-26, 2000. While SDE has a molecular weight of 881 Da, the SDE formulations disclosed herein have the surprising ability to penetrate and accumulate in the skin.

In various embodiments, a non-aqueous pharmaceutical composition for topical use comprises sebacoyl dinalbuphine ester (SDE) or a pharmaceutically acceptable salt thereof and at least one pharmaceutically acceptable excipient. Excipients may aid in lubricity, flowability, bioavailability, disintegration and may confer some form of antimicrobial function.

In various embodiments, the at least one pharmaceutically acceptable excipient is a penetration enhancer, for example, dimethyl isosorbide, diethylene glycol monoethyl ether, castor oil and oleyl alcohol. Other epidermal penetration enhancers known to those skilled in the art, include, but are not limited to, alcohols, alkanols, alkanones such as benzyl alcohol, decanol, ethanol, octanol, and propanol; polyols and esters thereof such as butanediol, ethylene glycol, glycerol, 1,2,6 hexanetriol, polyethylene glycol, propylene glycol monocaprylate, propylene glycol monolaurate and propylene glycol; fatty acids such as lauric acid, oleic acid, and valeric acid; fatty acid esters such as ethyl oleate, isopropyl myristate, and methylpropionate; amides and other nitrogenous compounds such as diethanolamine, dimethylacetamide, dimethylformamide, ethanolamine, 1-methyl-2-pyrrolidone, 2-pyrrolidone, triethanolamine, and urea; diisopropyl adipate, dimethyl isosorbide, ethers such as diethylene glycol monoethyl ether and diethylene glycol monomethyl ether; organic acids such as citric acid, salicylic acid, salicylates, and succinic acid; pyrrolidones such as 2-pyrrolidone; 1-substituted azacycloheptan-2-ones, such as 1-n-dodecylcyclazacycloheptan-2-one (laurocapram); sulfoxides such as decylmethylsulfoxide and dimethylsulfoxide, oleyl alcohol, castor oil.

In various embodiments, the at least one pharmaceutically acceptable excipient is a thickening agent, for example, polyethylene glycol (PEG) 4000, soft paraffin, stearic acid, hydroxypropyl cellulose (HPC), and others known to those skilled in the art. For instance, thickening agents may be microcrystalline cellulose, hydroxypropyl methylcellulose (HPMC), hydroxyethyl cellulose (HEC), methyl cellulose, ethylcellulose, Hydroxyethylcellulose, carboxymethylcellulose, polyvinylpyrrolidone, Carbopol (carbomer), sodium hyaluronate (hyaluronic acid), acacia, dextrin, polyethylene glycol 800˜8000, polysaccharides (such as dextrates, guar gum, and xanthan gum), saturated fatty acids with C12˜C22, and polyethers.

In various embodiments, the at least one pharmaceutically acceptable excipient is a solvent. The solvent may be, for example, PEG 400, diisopropyl adipate, benzyl benzoate, N-methyl-2-pyrrolidone (NMP), isopropyl myristate (IPM), Caprylic/Capric Triglyceride (Medium Chain Triglycerides), liquid paraffin, and others known to those skilled in the art. For instance, solvent may be alcohol, castor oil, 3-[(3-cholamidopropyl)dimethylammonio]-1-propane-sulfonate, cholesterol NF, cholic acid, citric acid, 3-cyclohexene-1-methanol, dehydrated alcohol, deoxycholic acid, diethylene glycol monoethyl ether, diisopropanolamine (1:9), a4-dimethyl-a-(4-methyl-3-pentenyl), ethoxydiglycol, ethoxylated alcohol, ethyl alcohol, ethylene glycol, fatty alcohol citrate, glycerin, 1-hexadecanol, 1,2,6-hexanetriol, hexylene glycol, hydroxypropyl betacyclodextrin, isopropyl alcohol, isopropyl myristate, isopropyl palmitate, lecithin, mineral oil, 2-methyl-1,3-propanediol, oleyl alcohol, phosphoric acid, polyethylene glycols, polyethylene glycol 200-600, polyethylene glycol 1000 monocetyl ether, polyethylene glycol monostearate, polyoxyl 20 cetostearyl ether, polyoxypropylene 15-stearyl ether, polysorbates, polysorbate 20/40/60/80, potassium hydroxide, propylene carbonate, propylene glycol, propylethylene glycol 4, neopentyl alcohol, SD alcohol 40, sodium lauryl sulfate, sorbitan monostearate, sorbitan stearate, taurodeoxycholic acid, triacetin, triethylene glycol, trimethylene glycol, dimethicone, petrolatum, propylene glycol dicaprylate/dicaprate, vegetable oils, and phenoxyethanol.

In various embodiments, the at least one pharmaceutically acceptable excipient is an antioxidant. In certain embodiments, the antioxidant is butylated hydroxytoluene (BHT). In other embodiments, the antioxidant is butylated hydroxyanisole (BHA), ethylenediaminetetraacetic acid (EDTA), propyl gallate, ascorbic acid, citric acid, ascorbyl palmitate, alpha-tocopherol, alpha-tocopherol acetate and others known to those skilled in the art. Examples include, but are not limited to, ascorbic acid polypeptide, ascorbyl dipalmitate, potassium metabisulfite, magnesium ascorbyl phosphate, propyl gallate sodium ascorbate, sodium metabisulfite, sodium ascorbyl/cholesteryl phosphate, sodium bisulfite, sodium erythorbate, sodium thiosulfate, vitamin E, tocopheryl nicotinate, and 3,4-dihydroxybenzoic acid.

In various embodiments, the non-aqueous pharmaceutical composition comprises a solvent, a thickening agent, and a penetration enhancer.

In various embodiments, the non-aqueous pharmaceutical composition comprises from about 0.1 to about 5 percent (w/w) SDE, from about 34 to about 85 percent (w/w) solvent, from about 0.8 to about 40 percent (w/w) thickening agent, and from about 0 to 65 percent (w/w) penetration enhancer.

In various embodiments, the non-aqueous pharmaceutical composition comprises from about 0.1 to about 2 percent (w/w) SDE, from about 0 to about 15 percent (w/w) dimethyl isosorbide, from about 44 to about 70 percent (w/w) PEG 400, from about 0 to about 10 percent (w/w) diethylene glycol monoethyl ether, about 15 percent (w/w) diisopropyl adipate, and about 16% (w/w) PEG 4000.

In various embodiments, the non-aqueous pharmaceutical composition comprises from about 0.1 to about 5.1 percent (w/w) SDE, from about 0 to about 15 percent (w/w) dimethyl isosorbide, from about 14 to about 65 percent (w/w) PEG 400, from about 10 to about 50 percent (w/w) diethylene glycol monoethyl ether, from about 15 to about 20 percent (w/w) diisopropyl adipate, and about 0.8 percent (w/w) hydroxypropylcellulose.

In various embodiments, the non-aqueous pharmaceutical composition comprises from about 0.1 to about 4.1 percent (w/w) SDE, about 15 percent (w/w) dimethyl isosorbide, about 14.1 percent PEG 400, about 50 percent (w/w) diethylene glycol monoethyl ether, about 20 percent (w/w) diisopropyl adipate, and about 0.8 percent (w/w) hydroxypropylcellulose.

In various embodiments, the non-aqueous pharmaceutical composition comprises from about 4 percent (w/w) SDE.

In various embodiments, the non-aqueous pharmaceutical composition comprises from about 0.1 to about 3.2 percent (w/w) SDE, from about 5 to about 25 percent (w/w) isopropyl myristate, from about 0 to about 10 percent (w/w) oleyl alcohol, from about 0 to about 20 percent (w/w) castor oil, about 5 to about 15 percent (w/w) Caprylic/Capric Triglyceride, about 0 to about 25 percent (w/w) liquid paraffin, about 10 to about 30 percent (w/w) diisopropyl adipate, about 26 to about 45 percent (w/w) soft paraffin, and from about 0 to about 8 percent (w/w) stearic acid.

In various embodiments, the non-aqueous pharmaceutical composition comprises from about 0.1 to about 2 percent (w/w) SDE, about 16 percent (w/w) isopropyl myristate, from about 14 percent (w/w) castor oil, about 10 percent (w/w) Caprylic/Capric Triglyceride, about 20 percent (w/w) diisopropyl adipate, about 32 percent (w/w) soft paraffin, and from about 0 to about 8 percent (w/w) stearic acid.

In various embodiments, the non-aqueous pharmaceutical composition comprises from about 0.5 to about 1.4 percent (w/w) SDE, from about 0 to about 15 percent (w/w) dimethyl isosorbide, from about 44 to about 70 percent (w/w) PEG 400, from about 0 to 10 percent (w/w) diethylene glycol monoethyl ether, about 15 percent (w/w) diisopropyl adipate, and about 16% (w/w) PEG 4000.

In various embodiments, the non-aqueous pharmaceutical composition comprises from about 1.3 to about 4.1 percent (w/w) SDE, from about 0 to about 15 percent (w/w) dimethyl isosorbide, from about 14 to about 65 percent (w/w) PEG 400, from about 10 to about 50 percent (w/w) diethylene glycol monoethyl ether, from about 15 to about 20 percent (w/w) diisopropyl adipate, and from about 0 to about 0.8 percent (w/w) hydroxypropylcellulose. In certain embodiments, the pharmaceutical composition further comprises from about 5 to 15 percent (w/w benzyl benzoate). In other embodiments, the pharmaceutical composition further comprises about 15 percent (w/w) NMP.

In some embodiments, the non-aqueous pharmaceutical composition comprises about 4.1 percent (w/w) SDE, about 15 percent (w/w) dimethyl isosorbide, 14.1 percent (w/w) PEG 400, about 50 percent (w/w) diethylene glycol monoethyl ether, about 20 percent (w/w) diisopropyl adipate, and about 0.8 percent (w/w) hydroxypropylcellulose.

In various embodiments, the non-aqueous pharmaceutical composition comprises from about 0.4 to about 1.5 percent (w/w) SDE, from about 5 to about 20 percent (w/w) isopropyl myristate, from about 0 to about 15 percent (w/w) castor oil, from about 0 to about 10 percent (w/w) oleyl alcohol, about 9.9 percent Caprylic/Capric Triglyceride, about 0 to about 25 percent (w/w) liquid paraffin, about 10 to about 20 percent diisopropyl adipate, about 32 percent (w/w) soft paraffin, and from about 0 to about 8 percent (w/w) stearic acid. In other embodiments, the pharmaceutical composition further comprises about 10 percent (w/w) benzyl benzoate.

In some embodiments, the non-aqueous pharmaceutical composition comprises from about 0.1 to about 2 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, from about 32 to about 37 percent (w/w) soft paraffin, and from about 0 to about 4 percent (w/w) stearic acid.

In some embodiments, the non-aqueous pharmaceutical composition comprises about 1 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, about 33 percent (w/w) soft paraffin, and about 4 percent (w/w) stearic acid.

In some embodiments, the non-aqueous pharmaceutical composition comprises about 1 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, and about 37 percent (w/w) soft paraffin.

In some embodiments, the non-aqueous pharmaceutical compositions disclosed herein further comprise an antioxidant.

In some embodiments, the non-aqueous pharmaceutical composition comprises a solvent, and/or a penetration enhancer.

In some embodiments, the non-aqueous pharmaceutical composition comprises from about 0.1 to about 1 percent (w/w) SDE, from about 79 to about 90 percent (w/w) solvent, and from about 9 to 20 percent (w/w) penetration enhancer.

In some embodiments, the non-aqueous pharmaceutical composition comprises from about 0.1 to about 1 percent (w/w) SDE, from about 10 to about 25 percent (w/w) isopropyl myristate, from about 9 to about 20 percent (w/w) castor oil, from about 5 to about 15 percent (w/w) Caprylic/Capric Trigylceride, from about 14 to about 30 percent (w/w) diisopropyl adipate, and from about 26-45 percent (w/w) liquid paraffin.

In some embodiments, the non-aqueous pharmaceutical composition comprises about 1 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, and from about 37 percent (w/w) liquid paraffin.

### Methods

The disclosure herein focuses, in part, on the discovery that topically administered SDE can penetrate the epidermis and dermis from certain formulations. Without being bound by theory, topical SDE penetrates the epidermis and dermis and accumulates therein where it is de-esterified causing the prolonged local release of nalbuphine in the skin. The disclosure herein is also focused on the therapeutic applications of topical SDE formulations, specifically the anti-pruritus activity of topical SDE administration.

In various embodiments, a method of treating disorders comprises topically administering a pharmaceutically effective amount of a pharmaceutical composition comprising SDE or a pharmaceutically acceptable salt thereof and at least one pharmaceutically acceptable excipient to a subject in need thereof.

In various embodiments, the disorders are selected from pruritus conditions, pain, or inflammatory conditions.

In various embodiments, the disorder is a pruritus condition. In various embodiments, the pruritus condition can be uremic pruritus, atopic dermatitis, prurigo, nervous dermatitis, aquagenic pruritus, atopic dermatitis, photosensitive dermatosis, idiopathic pruritus, Lichen simplex chronicus, prurigo nodularis, psoriasis, cholestatic pruritus, contact dermatitis, seborrheic dermatitis, autosensitization dermatitis, caterpillar dermatitis, eczema, asteatosis, senile pruritus cutaneous, insect sting, scabies, urticaria, herpes, impetigo, tinea, lichen, acne vulgaris, or visceral diseases complicated with pruritus, brachioradial pruritus, burn-induced pruritus, cancer-induced pruritus, neuropathic pruritus, morphine-induced pruritus, multiple sclerosis-associated pruritus, post-herpetic pruritus, bullous pemphigoid, Netherton syndrome, or itching from poison or any other stimulus. In other embodiments, the pruritus condition can be uremic pruritus, atopic dermatitis, or prurigo nodularis. In other embodiments, the methods disclosed herein can be used for the treatment of a subject suffering from a pruritic condition associated with an elevated substance P level.

In certain embodiments, the pharmaceutical compositions disclosed herein may be administered eight times a day, six times a day, four times a day, three times a day, twice a day, once a day, or every 2, 3, 4, 5, 6, 7 days, or longer. The pharmaceutical composition may also be administered on an appropriate schedule as determined by one skilled in the art.

In various embodiments, the administered pharmaceutical composition comprises a topical gel, topical ointment, topical lotion or topical cream.

In various embodiments, the method comprises a pharmaceutical composition that releases nalbuphine from SDE over the course of 2, 4, 6, 8, 12, 24, 48, 72 hours, or longer.

In various embodiments, the SDE concentration in the epidermis and dermis at 24 hours post administration is higher than the SDE concentration in the circulation.

In various embodiments, the methods disclosed herein alleviate or partially alleviate at least one symptom of pruritus.

In various embodiments, the method comprises administering the pharmaceutical compositions disclosed herein, wherein the subject does not experience opioid-related side effects or in which such side effects are reduced when compared with subjects to whom effective doses of opiate were administered systemically.

While illustrative embodiments are described herein, the scope of the invention includes any and all embodiments having equivalent elements, modifications, omissions, combinations (e.g., of aspects across embodiments), adaptations or alterations based on the present disclosure. The elements in the claims are to be interpreted based on the language employed in the claims and not limited to examples described in the specification or during the prosecution of the application, which examples are to be construed as non-exclusive. It is intended, therefore, that the specification and examples be considered as example only, with a true scope and spirit being indicated by the claims and their full scope of equivalents.

### EXAMPLES

### Example 1: Excipient Compatibility Test

Individual excipients were investigated to determine their compatibility with SDE (at 50° C. for 2 and 4 weeks) for potential use in a topical formulation. The results are presented in Table 1.

Significant reductions in SDE purity were observed for deionized water (at both pH 4 and 5) and Transcutol P. These reductions in SDE purity were potentially caused by hydrolysis and/or oxidation of the drug. The combination of BHT with Transcutol P was observed to reduce the degree of SDE degradation, when compared to Transcutol P alone.

TABLE 1

| | SDE purity (%) | | |
|---|---|---|---|
| | | 50° C. | |
| Excipients | T = 0 | 2 weeks | 4 weeks |
| water (pH 4) | 96.61 | 28.91 | 14.47 |
| water (pH 5) | 96.83 | 28.74 | 5.43 |
| PEG 400 (pH 4-5) | 100.00 | 94.01 | 96.97 |
| PEG 400 (pH 4-5) + BHT | 98.31 | 96.38 | 93.24 |
| Super refined PEG 400 (pH 4-5) | 100.00 | 93.17 | 92.23 |
| Transcutol P | 94.31 | 5.69 | 1.50 |
| Transcutol P + BHT | 96.90 | 14.65 | 4.21 |
| Castor oil | 100.00 | 80.20 | 75.71 |
| Diisopropyl adipate | 100.00 | 99.60 | 99.49 |
| Isopropyl myristate (IPM) | 100.00 | 99.51 | 85.75 |
| Miglyol 810 (Caprylic/Capric triglyceride) | 100.00 | 99.66 | 99.69 |

### Example 2: Pre-Formulation Experiments

A range of preliminary solvent systems (composition details are provided in Table 2) were prepared to investigate the short-term stability of SDE in these solvent systems. The results are presented in Table 3.

In SS1, SS11 and SS12, significant reductions in SDE purity were observed at 2 and 4 weeks of storage at 50° C. The results demonstrate that SDE is prone to degradation in aqueous solvent systems, thus, non-aqueous solvent systems are preferred for formulations comprising SDE.

Consistent SDE purity results following 2 weeks storage at 50° C. compared to T=0 were observed for SS6 (with a slight reduction at 4 weeks) despite the presence of 15% Transcutol P.

TABLE 2

| | Composition (% w/w) | | | | | |
|---|---|---|---|---|---|---|
| Excipients | SS1 | SS4 | SS6 | SS11 | SS12 | SS13 |
| Deionised water | 69.68 | — | — | 69.68 | 69.68 | — |
| (pH 4) | | | | | | |
| PEG 400 | — | 69.90 | 69.90 | 15.00 | 15.00 | 69.90 |
| SR-DMI | — | — | — | — | — | 15.00 |
| Transcutol P | 15.00 | 15.00 | 15.00 | — | 15.00 | — |
| DIPA | — | — | 15.00 | — | — | — |
| Glycerol | 15.00 | 15.00 | — | 15.00 | — | 15.00 |
| Methyl parabens | 0.2 | — | — | 0.2 | 0.2 | — |
| Propyl parabens | 0.02 | — | — | 0.02 | 0.02 | — |
| BHT | — | 0.1 | 0.1 | — | — | 0.1 |
| Propyl gallate | 0.1 | — | — | 0.1 | 0.1 | — |
| Total | 100 | 100 | 100 | 100 | 100 | 100 |
| Final pH of system | 4.0 | 4-5 | 4-5 | 4.0 | 4.0 | 4-5 |

TABLE 3

SDE purity (%)

50° C.

| Solvent system | T = 0 | 2 weeks | 4 weeks |
|---|---|---|---|
| SS1 | 99.75 | 45.12 | 16.06 |
| SS4 | 99.97 | 87.45 | 75.16 |
| SS6 | 100.00 | 95.11 | 90.66 |
| SS11 | 99.24 | 37.17 | 13.05 |
| SS12 | 99.48 | 45.40 | 15.62 |
| SS13 | 99.62 | 83.96 | 73.33 |

### Example 3: Non-Aqueous Polyethylene Glycol Ointment Formulations

Three Polyethylene Glycol (PEG) ointment formulations listed in Table 4 were prepared as follows. Butylated hydroxytoluene (BHT) was weighed into a vessel before the addition of the liquid excipients: super-refined dimethyl isosorbide (SR DMI), PEG 400, transcutol P, and diisopropyl adipate. The contents were stirred until the BHT had dissolved and the contents were visibly homogenous. SDE was then added into the mixture and stirred until visibly dissolved. The solid excipient, PEG 4000, was weighed into a separate container, heated in a water bath at 70° C., and stirred until a clear melt was observed. The clear melt was added to the liquid phase mixture which had been heated to 70° C., and the mixture was stirred until visually mixed. The formulation was removed from the water bath and stirred until cooled to room temperature. For each formulation, active formulation (with SDE) and placebo formulation (without SDE) were both prepared, the percent SDE in each active formulation is shown in Table 4. The saturated solubility of SDE in each formulation was also determined. When determining the saturated solubility, thickening agent and antioxidant, PEG 4000 and BHT, were not included in the formulation.

TABLE 4

| Formulation No. | PO4 | PO6 | PO7 |
|---|---|---|---|
| SR DMI | — | — | 15 |
| PEG 400 | 68.9 | 59.9 | 44.9 |
| Transcutol P* | — | 9 | 9 |
| Diisopropyl adipate | 15 | 15 | 15 |
| PEG 4000 | 16 | 16 | 16 |
| BHT | 0.1 | 0.1 | 0.1 |
| SDE | 0.51 | 0.66 | 1.36 |
| (% w/w) | | | |
| Saturated solubility of | 0.76 | 0.98 | 2.02 |
| (% w/w) | | | |

*Diethylene glycol monoethyl ether

### Example 4: Non-Aqueous Gel Formulations

Seven non-aqueous gel formulations listed in Table 5 were prepared as follows. BHT was weighed into a vessel before the addition of the liquid excipients: SR DMI, PEG400, transcutol P, diisopropyl adipate, benzyl benzoate and N-methyl-2-pyrrolidone (NMP). The contents were stirred until the BHT was dissolved and the contents were visibly homogenous. SDE was then added into the mixture and stirred until visibly dissolved. The thickening agent, hydroxypropylcellulose (HPC HF), was then added into the liquid phase and then stirred until visibly solvated. For each formulation, active formulation (with SDE) and placebo formulation (without SDE) were both prepared, the percent SDE in each active formulation is shown in Table 5. The saturated solubility of SDE in each formulation was also determined and is shown in Table 5. When determining the saturated solubility, thickening agent and antioxidant, HPC HF and BHT, were not included in the formulation.

The saturated solubility of SDE in the NA8 formulation is 5.12% (w/w), which is the highest among the seven non-aqueous gel formulations.

TABLE 5

| Formulation No. | NA7 | NA8 | NA10 | NA12 | NA14 | NA15 | NA16 |
|---|---|---|---|---|---|---|---|
| SR DMI | 15 | 15 | — | — | — | 15 | 15 |
| PEG 400 | 39.1 | 14.1 | 54.1 | 54.1 | 64.1 | 52.16 | 52.34 |
| Transcutol P | 25 | 50 | 15 | 15 | 15 | 15 | 10 |
| Diisopropyl adipate | 20 | 20 | 15 | 15 | 15 | 15 | 20 |
| Benzyl benzoate | — | — | 15 | — | 5 | — | — |
| NMP | — | — | — | 15 | — | — | — |
| BHT | 0.1 | 0.1 | 0.1 | 0.1 | 0.1 | 0.1 | 0.1 |
| HPC HF | 0.8 | 0.8 | 0.8 | 0.8 | 0.8 | 0.8 | 0.8 |
| SDE | 2.62 | 4.08 | 1.97 | 2.17 | 1.34 | 1.94 | 1.76 |
| (% w/w) | | | | | | | |
| Saturated solubility | 3.27 | 5.10 | 2.46 | 2.71 | 1.67 | 2.43 | 2.2 |
| of SDE | | | | | | | |
| (% w/w) | | | | | | | |

### Example 5: Non-Aqueous Ointment Formulations

Five ointment formulations listed in Table 6 were prepared as follows. BHT was weighed into a vessel before the addition of the liquid excipients: castor oil, isopropyl myristate (IPM), oleyl alcohol, Miglyol 810, liquid paraffin, diisopropyl adipate, and benzyl benzoate. The contents were stirred until the BHT had dissolved and the contents were visibly homogenous. SDE was then added into the mixture and stirred until visibly dissolved to form a liquid phase mixture. The solid excipients, soft paraffin, and stearic acid, were weighed into a separate container, heated in a water bath at 70° C., and stirred until a clear melt was observed. The clear melt was added to the liquid phase mixture which had been heated to 70° C., and the mixture was stirred until visually mixed. The formulation was removed from the water bath and stirred until cooled to room temperature. For each formulation, active formulation (with SDE) and placebo formulation (without SDE) were both prepared; the percent SDE in each active formulation is shown in Table 6. The saturated solubility of SDE in each formulation was also determined and shown in Table 6. When determining the saturated solubility, thickening agent and antioxidant, soft paraffin, stearic acid, and BHT, were not included in the formulation.

TABLE 6

| Formulation No. | OO1 | OO2 | OO3 | OO4 | OO5 |
|---|---|---|---|---|---|
| Castor Oil | — | 5 | 14 | 14 | 14 |
| IPM | 5 | 15 | 16 | 16 | 16 |
| Oleyl Alcohol | 10 | 10 | 10 | — | — |
| Miglyol 810 | 9.9 | 9.9 | 9.9 | 9.9 | 9.9 |
| Liquid Paraffin | 25 | 10 | — | — | — |
| Diisopropyl adipate | 10 | 10 | 10 | 10 | 20 |
| soft paraffin | 32 | 32 | 32 | 32 | 32 |
| Stearic Acid | 8 | 8 | 8 | 8 | 8 |
| Benzyl benzoate | — | — | — | 10 | — |
| BHT | 0.1 | 0.1 | 0.1 | 0.1 | 0.1 |
| SDE | 0.45 | 0.56 | 0.94 | 1.5 | 0.99 |
| (% w/w) | | | | | |
| Saturated solubility of SDE | 0.94 | 1.17 | 1.96 | 3.12 | 2.06 |
| (% w/w) | | | | | |

### Example 6: Macroscopic Appearance of Certain Formulations

The ten formulations listed in Table 7 were prepared according to Examples 3-5. For each formulation, active formulation (with SDE) and placebo formulation (without SDE) were both prepared, the percent SDE in each active formulation is shown in Table 7.

TABLE 7

| Formulation No. | PO7 | OO3 | OO4 | OO5 | NA7 | NA8 | NA10 | NA12 | NA14 | NA15 |
|---|---|---|---|---|---|---|---|---|---|---|
| Castor Oil | — | 14 | 14 | 14 | — | — | — | — | — | — |
| IPM | — | 16 | 16 | 16 | — | — | — | — | — | — |
| Oleyl Alcohol | — | 10 | — | — | — | — | — | — | — | — |
| Miglyol 810 | — | 9.9 | 9.9 | 9.9 | — | — | — | — | — | — |
| SR DMI | 15 | — | — | — | 15 | 15 | — | — | — | 15 |
| PEG 400 | 44.9 | — | — | — | 39.1 | 14.1 | 54.1 | 54.1 | 54.1 | 52.16 |
| Transcutol P | 9 | — | — | — | 25 | 50 | 15 | 15 | 15 | 15 |
| Diisopropyl | 15 | 10 | 10 | 20 | 20 | 20 | 15 | 15 | 15 | 15 |
| adipate | | | | | | | | | | |
| PEG 4000 | 16 | — | — | — | — | — | — | — | — | — |
| Soft Paraffin | — | 32 | 32 | 32 | — | — | — | — | — | — |
| Stearic Acid | — | 8 | 8 | 8 | — | — | — | — | — | — |
| Benzyl benzoate | — | — | 10 | — | — | — | 15 | — | 5 | — |
| NMP | — | — | — | — | — | — | — | 15 | — | — |
| BHT | 0.1 | 0.1 | 0.1 | 0.1 | 0.1 | 0.1 | 0.1 | 0.1 | 0.1 | 0.1 |
| HPC HF | — | — | — | — | 0.8 | 0.8 | 0.8 | 0.8 | 0.8 | 0.8 |
| SDE | 1.36 | 0.94 | 1.5 | 0.99 | 2.62 | 4.08 | 1.97 | 2.17 | 1.34 | 1.94 |
| (% w/w) | | | | | | | | | | |

The macroscopic observations of the 10 selected formulations at time zero (t=0) have been summarized in Table 8A and 8B. No crystals of SDE were observed in any of the formulations and no differences were observed between any of the active and placebo formulations following storage for 2 or 4 weeks at 2-8° C. and 25° C. (Table 8A). No crystals of SDE were observed under the microscope with polarized or non-polarized light at any time point in any of the formulations. No differences were observed following storage for 2 or 4 weeks at 2-8° C. and 25° C. (Table 8B).

TABLE 8A

| | Macroscopy of developed formulations | | | | | | | | | |
|---|---|---|---|---|---|---|---|---|---|---|
| | | | T = 2 weeks | | | | T = 4 weeks | | | |
| | T = 0 | | 2-8° C. | | 25° C. | | 2-8° C. | | 25° C. | |
| Formulation | P | A | P | A | P | A | P | A | P | A |

| PO7 | Off-white, | Off-white, | No | No | No | No |
|---|---|---|---|---|---|---|
| OO3 | opaque, | opaque, | change | change | change | change |
| OO4 | medium-high | medium-high | from T = 0 | from T = 0 | from T = 0 | from T = 0 |
| OO5 | viscosity ointment | viscosity ointment | | | | |
| NA3 | Clear, colorless, | Clear, colorless, | | | | |
| NA7 | medium-low | medium-low | | | | |
| NA8 | viscosity gel | viscosity gel | | | | |
| NA10 | | | | | | |
| NA12 | | | | | | |
| NA14 | | | | | | |

P: placebo formulation;
A: active formulation

TABLE 8B

| | Microscopy of developed formulations | | | | | | | | | |
|---|---|---|---|---|---|---|---|---|---|---|
| | | | T = 2 weeks | | | | T = 4 weeks | | | |
| | T = 0 | | 2-8° C. | | 25° C. | | 2-8° C. | | 25° C. | |
| Formulation | P | A | P | A | P | A | P | A | P | A |
| PO7 | X | X | X | X | X | X | X | X | X | X |
| OO3 | X | X | X | X | X | X | X | X | X | X |
| OO4 | | | | | | | | | | |
| OO5 | | | | | | | | | | |
| NA3 | X | X | X | X | X | X | X | X | X | X |
| NA7 | | | | | | | | | | |
| NA8 | | | | | | | | | | |
| NA10 | | | | | | | | | | |
| NA12 | | | | | | | | | | |
| NA14 | | | | | | | | | | |

X = No SDE crystals observed under polarized and non-polarized light

P: placebo formulation; A: active formulation

### Example 7: Non-Aqueous Ointment Formulation II

The eight ointment formulations listed in Table 9 were prepared according to Example 5. For each formulation, active formulation (with SDE) and placebo formulation (without SDE) were both prepared, the percent SDE in each active formulation is shown in Table 9. These formulations were stored in glass vials at 4° C. and 25° C. for two weeks.

TABLE 9

| Formulation No. | P1 | P2 | P3 | P4 | P5 | P6 | P7 | P8 |
|---|---|---|---|---|---|---|---|---|
| Castor Oil | 9 | 20 | 9 | 20 | 9 | 20 | 9 | 20 |
| IPM | 10 | 18 | 25 | 20 | 10 | 20 | 25 | 20 |
| Miglyol 810 | 5 | 15 | 5 | 10 | 5 | 10 | 5 | 10 |
| Diisopropyl adipate | 30 | 20 | 25 | 14 | 30 | 23 | 25 | 14 |
| Soft paraffin | 0 | 0 | 0 | 0 | 45 | 26 | 29 | 35 |
| Stearic Acid | 0 | 0 | 0 | 0 | 0 | 6 | 0 | 0 |
| Liquid paraffin | 45 | 26 | 35 | 35 | 0 | 0 | 0 | 0 |
| SDE | 1 | 1 | 1 | 1 | 1 | 1 | 1 | 1 |
| (% w/w) | | | | | | | | |

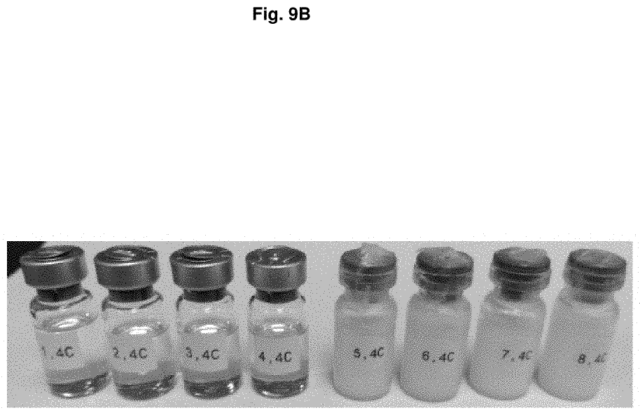

The visual or microscopic observations of the eight formulations after two weeks have been summarized in Table 10. For formulations P1-P4, the appearance of the formulations maintained clear after 2 weeks storage at 25° C. and 4° C., shown in FIGS. 9A and FIG. 9B, respectively. No precipitates or crystals of SDE were observed. For formulation P5-P8, the appearance of the formulation were non-transparent white ointment. No crystals of SDE were observed under the microscope with non-polarized light.

TABLE 10

| | | | | | | | |
|---|---|---|---|---|---|---|---|
| P1 | P2 | P3 | P4 | P5 | P6 | P7 | P8 |

4° C. for 2 weeks

| Visual | Clear solution | | | | White* | | | |
|---|---|---|---|---|---|---|---|---|
| observation | | | | | | | | |
| Microscopic | Pass | pass | pass | pass | Pass^{#} | Pass^{#} | Pass^{#} | Pass^{#} |
| observation | | | | | | | | |

25° C. for 2 weeks

| | | | | | | | | |
|---|---|---|---|---|---|---|---|---|
| Microscopic | pass | pass | pass | pass | Pass^{#} | Pass^{#} | Pass^{#} | Pass^{#} |
| observation | | | | | | | | |

*non-transparent white ointment
^{#}No SDE crystals observed under non-polarized light

### Example 8: Ex Vivo Skin Drug Permeation and Penetration

The ten formulations listed in Table 7 were used to evaluate the penetration of drug into human skin using the MedFlux-HT™ diffusion cell system. In this study, human skins with about 500±50 μm thickness were fixed on the MedFlux-HT diffusion cell system. About 10 mg of test formulation was applied to the epidermal side of the human skin, and a continued flow of receiver fluid was place at the opposite side of the skin. Because SDE is rapidly converted into nalbuphine by the esterases in the biological environment, both nalbuphine and SDE concentration were measured to access the drug permeation status. The concentration of nalbuphine and SDE in receiver fluid indicated the amount of drug that had penetrated through the human skin.

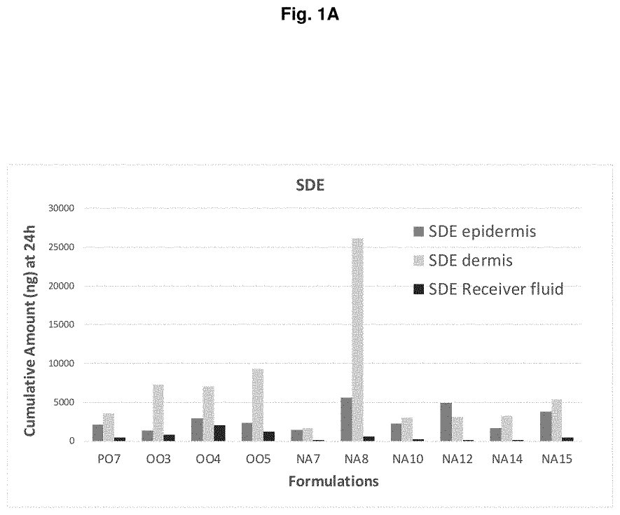

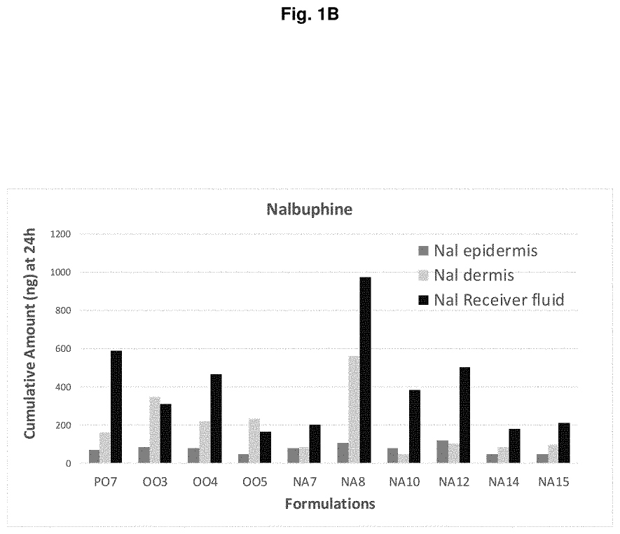

Tissue levels of SDE and nalbuphine in the dermis and epidermis, and the amount of SDE and nalbuphine in the receiver fluids over 24 hours were determined and are shown in FIG. 1A and FIG. 1B. SDE and nalbuphine were both detected in receiver fluid, dermis, and epidermis. The cumulative amounts of SDE and nalbuphine in receiver fluid recovered at 24 hours were 89-1978 ng/cm² and 169-978 ng/cm², respectively. These results indicate that SDE, with a molecular weight at 881.1 Da, which greatly exceed the general known allowable molecular size for skin permeation (Naik et al., Pharm Sci Technol Today, 2000;3:318-26), can penetrate through the skin when administered in each of the 10 tested formulations.

As a trend, NA8 and all three ointment formulations (OO3, OO4 and OO5) had the highest levels of SDE in the receiver fluid and dermis, and the highest levels of nalbuphine in the dermis. The non-aqueous gel and the non-aqueous ointment formulations may both be suitable formulations for treating pruritus.

### Example 9: Analysis of Skin Distribution

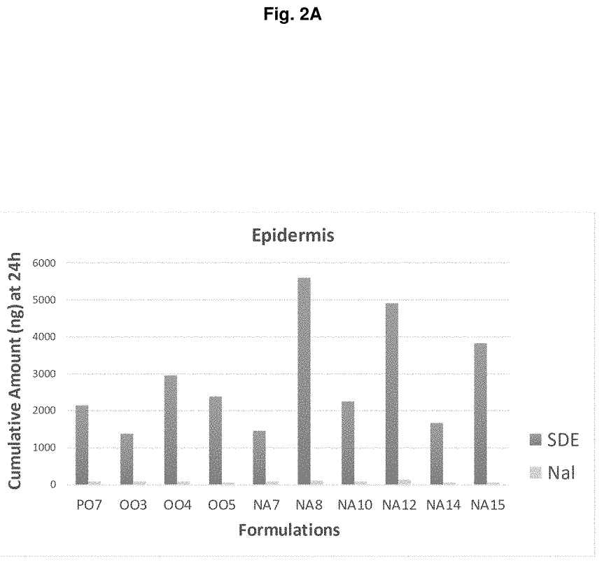

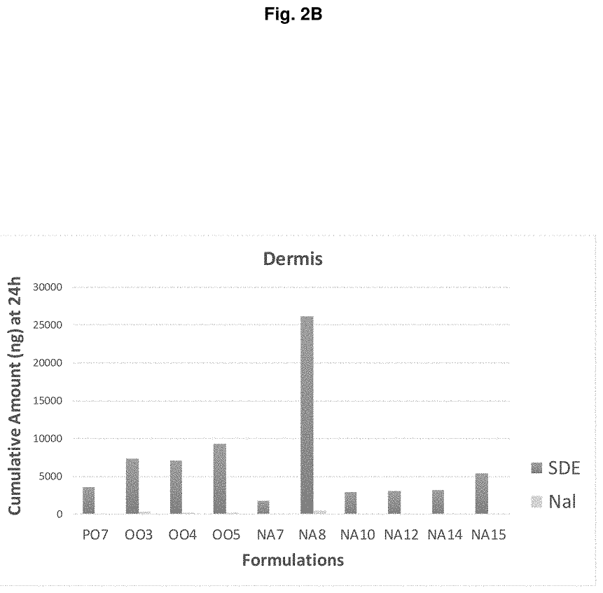

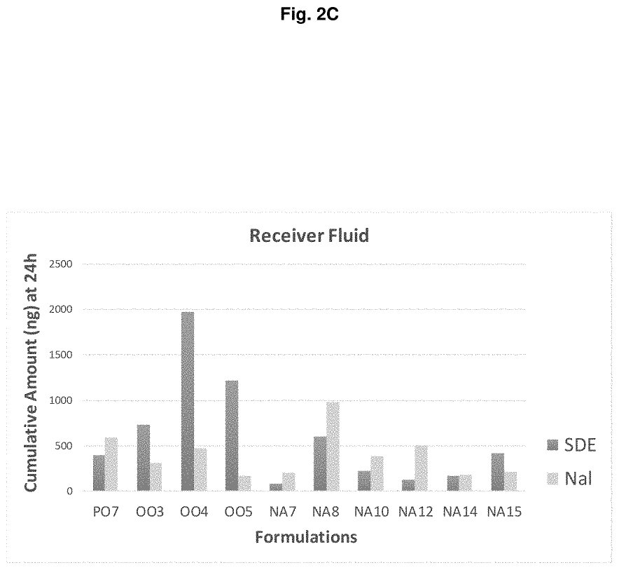

A comparison of the accumulated amount of SDE and nalbuphine in epidermis, dermis, and receiver fluid is shown in FIGS. 2A, 2B, and 2C, respectively.

The concentrations of SDE in dermis and epidermis were much greater than the concentrations of nalbuphine. A 10 fold difference was observed. (FIGS. 2A and 2B). On the other hand, the concentration of SDE in receiver fluid was comparable to the concentration of nalbuphine (FIG. 2C). The data from FIG. 1B also indicates that nalbuphine concentrations in the receiver fluid were greater than those in epidermis and dermis, a trend that was observed within all formulations.

By converting the detected accumulated amount of SDE/Nalbuphine into concentration, the relative concentrations of SDE and Nalbuphine in skin versus receiver fluid were calculated and are displayed in Table 11. The ratio of SDE concentration between skin and receiver fluid was significantly higher than that of Nalbuphine, indicating a low passive diffusion rate of SDE from skin to circulation.

High concentrations of nalbuphine in the blood lead to side effects such as sedation, dizziness, nausea, and vomiting. The results suggest that when SDE is hydrolyzed by esterases in the skin, nalbuphine penetrates through the skin and enters into the circulation in a relatively rapid manner. In contrast, SDE penetrates through the skin and enters into the circulation at a slower rate than nalbuphine. SDE, as a nalbuphine prodrug, may be retained in the dermis and epidermis for a longer period of time compared to nalbuphine, and may also avoid the rapid penetration of nalbuphine into the circulation. Thus, the adverse effects caused by systemic nalbuphine may be avoided. These characteristics suggest that the topical application of SDE may avoid many side effects typically associated with administering opiates.

TABLE 11

| Formulation No. | PO7 | NA7 | NA8 | NA10 | NA12 | NA14 | NA15 | OO3 | OO4 | OO5 |
|---|---|---|---|---|---|---|---|---|---|---|
| SDE concentration in skin | 114200 | 63560 | 634320 | 104440 | 160280 | 97720 | 184340 | 173740 | 200700 | 233160 |
| (mg/mL)* | | | | | | | | | | |
| SDE concentration in | 46.4 | 8.7 | 107.5 | 21.5 | 15.7 | 12.0 | 38.0 | 88.0 | 151.0 | 34.4 |
| receiver fluid (mg/mL) | | | | | | | | | | |
| SDE concentration ratio | 2464 | 7316 | 5900 | 4862 | 10206 | 8115 | 4850 | 1973 | 1329 | 6776 |
| (skin versus receiver | | | | | | | | | | |
| fluid) | | | | | | | | | | |
| Nalbuphine concentration | 4750 | 3374 | 13454 | 2622 | 4460 | 2710 | 2944 | 8680 | 6076 | 5722 |
| in skin (mg/mL)* | | | | | | | | | | |
| Nalbuphine concentration | 38.5 | 22.4 | 126.2 | 33.5 | 46.1 | 17.0 | 19.0 | 55.0 | 60.3 | 16.1 |
| in receiver fluid (mg/mL) | | | | | | | | | | |
| Nalbuphine | 123 | 151 | 107 | 78 | 97 | 160 | 155 | 158 | 101 | 355 |
| concentration ratio (skin | | | | | | | | | | |
| versus receiver fluid) | | | | | | | | | | |

*(The total amount of SDE/Nalbuphine in epidermis and dermis)/skin volume

### Example 9: Stability of Candidate Formulations

To understand the stability of the formulations, NA8 and OO5 were prepared according to Example 4 and 5. The tested samples were place in 40° C./75% RH stability chamber for 18 days and analyzed by ultra-performance liquid chromatography (UPLC). When determining the stability, antioxidant, BHT, was not included in the formulations.

UPLC analysis was conducted by using the Xbridge Shield RP18, 3.5 um, 4.6*250 mm column under the following conditions:

| Flow rate | 0.45 | mL/min |
|---|---|---|
| Run time | 85 | minutes |
| Detector | UV 280 nm | |
| Column temperature | 40° | C. |
| Sample temperature | 15° | C. |

| | |
|---|---|
| Mobile phase | Buffer A*/methanol gradient |

*Buffer A: acetate buffer.

The impurity profiles of each of the formulations are summarized in Table 12. Data showed that the stability of SDE in the ointment-based formulation was amenable to commercial use.

TABLE 12

| Impurities | NA8 | OO5 |
|---|---|---|
| Imp 1 | 2.20% | 0.13% |
| Imp 2 | 0.42% | 0.45% |
| Imp 3 | 3.20% | 0.12% |
| Imp 4 | 10.52% | 2.39% |

### Example 10: Anti-Pruritus Activity of SDE Via Subcutaneous Injection

The time-course and antipruritic effects of SDE via subcutaneous (SC) injection were studied in the mouse substance P-induced scratching model to evaluate the effect of SDE's antipruritic effect. In this study, SDE was dissolved in an oily formulation, with a concentration at 75 mg/mL.

Substance P, the endogenous ligand for the neurokinin-1 (NK-1) receptor, is a significant mediator of pruritus. See Andoh et al., J. Pharmacol. Exp. Ther., 286:1140-5, 1998. Intradermal injection of substance P elicits an itch sensation in human subjects, and an associated itch response in mice. See Amatya et al., Skin Pharmacol. Physiol., 23:133-138, 2010.

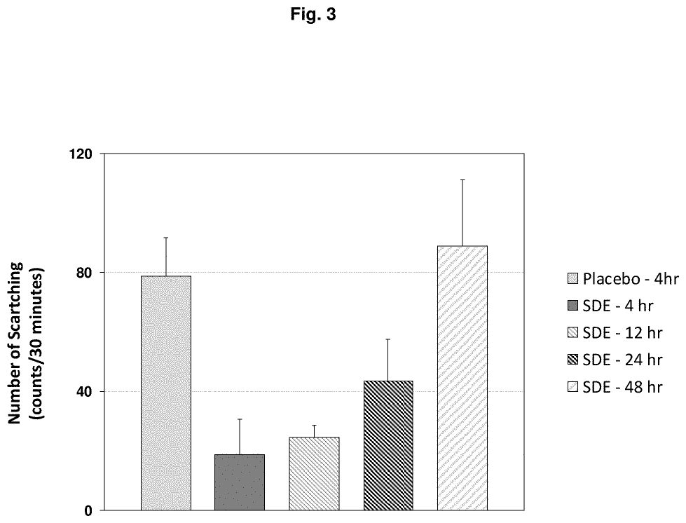

Intradermal injection of substance P successfully induced an average of 78.7±12.9 scratching bouts over a 30-minute observation period compared to SC injection of placebo control. Compared to the placebo control group, SDE (administered SC at 10 mL/kg) was associated with significant reduction of substance P-induced scratching behaviors at 4 hours (18.8±11.8 bouts) and 12 hours (24.5±4.1 bouts) post treatment. The attenuation of scratching responses diminished over time during the following 36 hours, as evidenced by a 45% suppressive effect at 24 hours and the loss of activity at 48 hours (FIG. 3). Furthermore, signs of depression, tremor and hypothermia were observed, as well as deaths and moribund animals at 24 and 48 hours, resulting in a 7.7% and 35.7% mortality rate, respectively (data not shown). Together, SDE demonstrated an antipruritic activity over 4 to 24 hours post SC administration. However, SC administration of SDE would cause high concentration of nalbuphine in the circulation and induce serious adverse effects.

### Example 11: Anti-Pruritus Activity of SDE Via Topical Administration—Non-Aqueous Gel Formulation

The time-course and antipruritic effects of the NA8 formulation via topical application were investigated in the mouse substance P-induced scratching model. Groups of 10 or 20 male ICR mice weighing 23±3 grams were used. One day before testing, hair was removed at the substance P injection sites. On the testing day, NA8 active formulation and NA8 placebo formulation (NA8 Vehicle) were applied topically on the rostral portion of the back (2×2 cm region) at 2 or 4 hours prior to substance P challenge. Substance P (250 nmol/site) was injected intradermally (ID) in a volume of 50 μL/site into the rostral portion of the back (the same region as test compound application). As reference, nalbuphine HCl was injected subcutaneously on the lower back region (different site as Substance P challenge). The scratching behavior was recorded for 30 minutes by visual observation immediately after substance P injection.

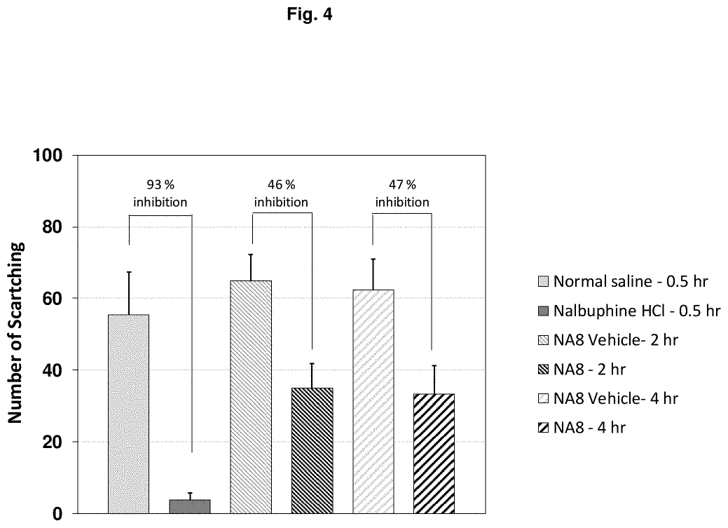

Normal saline and the two NA8 Vehicle groups had approximately 56-65 scratching events during a 30-minute observation period following substance P challenge (FIG. 4). Nalbuphine HCl (30 mg/kg, SC) significantly reduced substance P-evoked scratching behaviors at 0.5 hour post treatment as compared to the respective control. In addition, topical application of NA8 markedly demonstrated 46%-47% of inhibition on scratching behaviors at both 2 hours and 4 hours post treatment time points as compared to the respective NA8 Vehicle control, indicating the antipruritic activity against substance P challenge by topical application of SDE formulation.

### Example 12: Non-Systemic Anti-Pruritus Effects of Topical Application—Non-Aqueous Gel Formulation

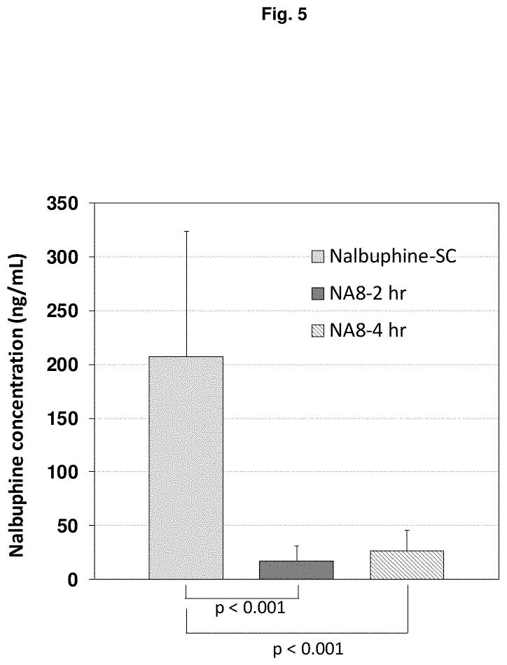

Immediately after the scratching observation of Example 11, whole blood was collected from each mouse via the vena cava, and the nalbuphine concentration in each sample was determined. The average whole blood concentration of nalbuphine for each condition are shown in FIG. 5.

SC injection of nalbuphine HCl at 30 mg/kg, SC at 0.5 hour is known to be an effective dose for pruritus treatment via systemic administration (Hawi A et al., Nalbuphine attenuates itch in the substance-P induced mouse model. Acta Derm Venereol, 2013, 93:634). The data from Example 10 and Example 11 indicate that the effective systemic concentration of nalbuphine when this dose was administered was about 206.84 ng/m L.

The whole blood concentrations of nalbuphine at 2 hours and 4 hours after topical administration of NA8 were 16.96 ng/mL and 26.55 ng/mL, respectively. The whole blood concentration of nalbuphine after topical administration of NA8 was statistically lower than the effective systemic concentration for nalbuphine's anti-pruritus effect. These results indicate that the anti-pruritus effect of the NA8 formulation was non-systemic, and that the topical formulation may work via targeting the peripheral opioid receptor system residing in the dermis and epidermis.

### Example 13: Ex Vivo Skin Drug Permeation and Penetration—Non-Aqueous Ointment Formulation

The four ointment formulations listed in Table 13 were prepared according to the method described in Example 5. The saturated solubility of SDE in each formulation was also determined and is shown in Table 13. When determining the saturated solubility, thickening agent and antioxidant, soft paraffin, stearic acid and BHT, were not included in the formulation.

These formulations were used to evaluate the penetration of drug into human skin using the MedFlux-HT™ diffusion cell system according to Example 8.

TABLE 13

| Formulation No. | TA-1 | TA-2 | TA-3 | TA-4 |
|---|---|---|---|---|
| Castor Oil | 14 | 14 | 14 | 14 |
| IPM | 18 | 18 | 18 | 18 |
| Miglyol 810 | 10 | 10 | 10 | 10 |
| Diisopropyl adipate | 20 | 20 | 20 | 20 |
| Soft paraffin | 32.89 | 35.89 | 36.89 | 0 |
| Liquid paraffin | 0 | 0 | 0 | 36.89 |
| Stearic Acid | 4 | 1 | 0 | 0 |
| VitC palmitate | 0.01 | 0.01 | 0.01 | 0.01 |
| BHT | 0.1 | 0.1 | 0.1 | 0.1 |
| SDE | 1 | 1 | 1 | 1 |
| (% w/w) | | | | |
| Saturated solubility of SDE | 1.63 | 1.63 | 1.63 | 1.63 |
| (% w/w) | | | | |

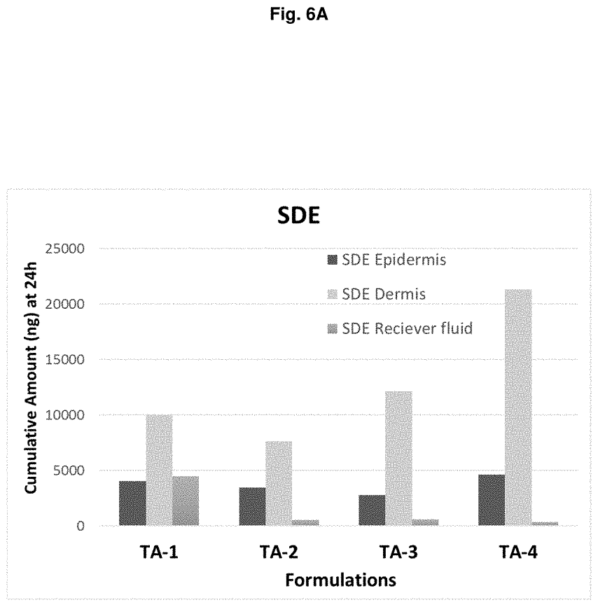

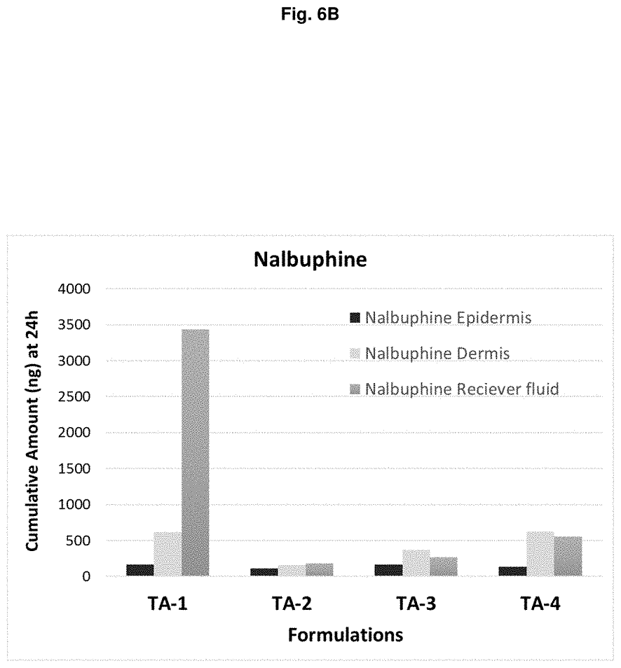

Tissue levels of SDE and nalbuphine in the dermis and epidermis, and the amount of SDE and nalbuphine in the receiver fluids over 24 hours were determined and are shown in FIG. 6A and FIG. 6B. SDE and nalbuphine were both detected in receiver fluid, dermis, and epidermis. The cumulative amounts of SDE and nalbuphine in receiver fluid recovered at 24 hours were 345-4480 ng/cm² and 131-3432 ng/cm², respectively. These results indicate that SDE can penetrate through the skin when administered in each of the four tested formulations.

The concentrations of SDE in dermis and epidermis were much greater than the concentrations of nalbuphine. On the other hand, the concentration of SDE in receiver fluid was comparable to the concentration of nalbuphine. The trend observed in Example 9 was also observed in each of the four tested formulations.

### Example 14: Anti-Pruritus Activity of SDE Via Topical Administration—Non-Aqueous Ointment Formulation

The time-course and antipruritic effects of the Ointment formulations via topical application were investigated in the mouse substance P-induced scratching model according to Example 11. On the testing day, the tested formulations (TA-1, TA-2, TA-3, and TA-4) were applied topically on the rostral portion of the back (2×2 cm region) at 4 hours prior to substance P challenge. 15 male ICR mice were used for each group. As reference, nalbuphine HCl was injected subcutaneously on the lower back region (different site as Substance P challenge). The scratching behavior was recorded for 30 minutes by visual observation immediately after substance P injection.

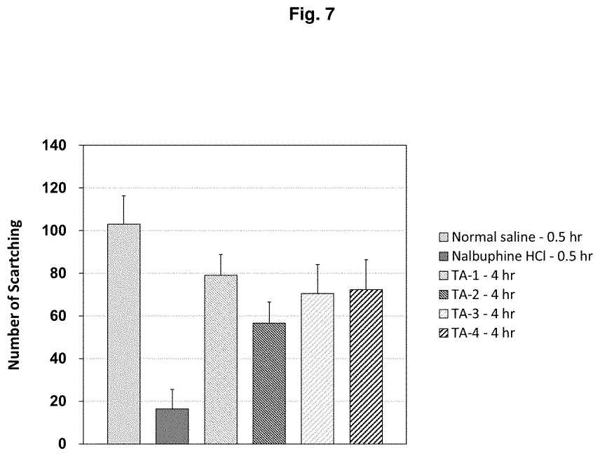

Normal saline had approximately 100 scratching events during a 30-minute observation period following substance P challenge (FIG. 7). Nalbuphine HCl (30 mg/kg, SC) significantly reduced substance P-evoked scratching behaviors at 0.5 hour post treatment as compared to the respective control. In addition, topical application of TA-1, TA-2, TA-3 and TA-4 markedly demonstrated ˜20% of inhibition on scratching behaviors at 4 hours post treatment time points as compared to normal saline control, indicating the antipruritic activity against substance P challenge by topical application of the tested formulations.

### Example 15: Non-Systemic Anti-Pruritus Effects of Topical Application—Non-Aqueous Ointment Formulation

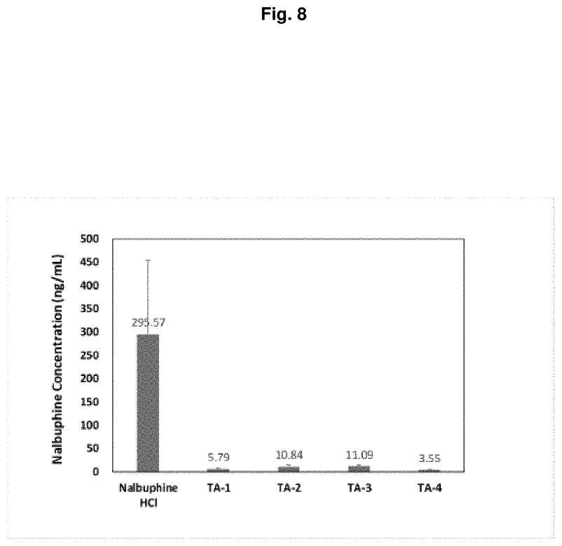

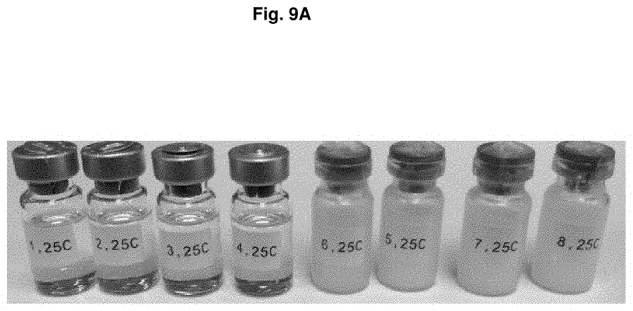

Immediately after the scratching observation of Example 14, whole blood was collected from each mouse via the vena cava, and the nalbuphine concentration in each sample was determined. The average whole blood concentration of nalbuphine for each condition are shown in FIG. 8.

The whole blood concentrations of nalbuphine at 4 hours after topical administration of TA-1, TA-2, TA-3 and TA-4 were about 3.55 ng/mL to 11.09 ng/m L. The whole blood concentration of nalbuphine after topical administration of TA-1, TA-2, TA-3 and TA-4 were statistically lower than the effective systemic concentration for nalbuphine's anti-pruritus effect. These results indicate that the anti-pruritus effect of the non-aqueous ointment formulations were non-systemic, and that these topical formulations may work via targeting the peripheral opioid receptor system residing in the dermis and epidermis.

We claim:

1. A non-aqueous pharmaceutical composition for topical use comprising sebacoyl dinalbuphine ester (SDE) or a pharmaceutically acceptable salt thereof and at least one pharmaceutically acceptable excipient.

2. The pharmaceutical composition of claim 1, wherein at least one pharmaceutically acceptable excipient is a penetration enhancer.

3. The pharmaceutical composition of claim 2, wherein the penetration enhancer is selected from dimethyl isosorbide, diethylene glycol monoethyl ether, castor oil, and oleyl alcohol.

4. The pharmaceutical composition of claim 1, wherein at least one pharmaceutically acceptable excipient is a thickening agent.

5. The pharmaceutical composition of claim 4, wherein the thickening agent is selected from PEG 4000, soft paraffin, hydroxypropyl cellulose (HPC), and stearic acid.

6. The pharmaceutical composition of claim 1, wherein at least one pharmaceutically acceptable excipient is a solvent.

7. The pharmaceutical composition of claim 6, wherein the solvent is selected from PEG 400, diisopropyl adipate, benzyl benzoate, N-methyl-2-pyrrolidone, isopropyl myristate (IPM), caprylic/capric triglyceride, and liquid paraffin.

8. The pharmaceutical composition of claim 1, wherein at least one pharmaceutically acceptable excipient is an antioxidant.

9. The pharmaceutical composition of claim 8, wherein the antioxidant is selected from butylated hydroxytoluene (BHT), butylated hydroxyanisole (BHA), ethylenediaminetetraacetic acid (EDTA), propyl gallate, ascorbic acid, citric acid, ascorbyl palmitate, alpha-tocopherol, and alpha-tocopherol acetate.

10. The pharmaceutical composition of claim 1, comprising a solvent, a thickening agent, and a penetration enhancer.

11. The pharmaceutical composition of claim 10, comprising from about 0.1 to about 5 percent (w/w) SDE, from about 34 to about 85 percent (w/w) solvent, from about 0.8 to about 40 percent (w/w) thickening agent, and from about 0 to 65 percent (w/w) penetration enhancer.

12. The pharmaceutical composition of claim 1, comprising from about 0.1 to about 2 percent (w/w) SDE, from about 0 to about 15 percent (w/w) dimethyl isosorbide, from about 44 to about 70 percent (w/w) PEG 400, from about 0 to 10 percent (w/w) diethylene glycol monoethyl ether, about 15 percent (w/w) diisopropyl adipate, and about 16% (w/w) PEG 4000.

13. The pharmaceutical composition of claim 1, comprising from about 0.1 to about 5.1 percent (w/w) SDE, from about 0 to about 15 percent (w/w) dimethyl isosorbide, from about 14 to about 65 percent (w/w) PEG 400, from about 10 to about 50 percent (w/w) diethylene glycol monoethyl ether, from about 15 to about 20 percent (w/w) diisopropyl adipate, and about 0.8 percent (w/w) hydroxypropylcellulose.

14. The pharmaceutical composition of claim 13, comprising about 0.1-4 percent (w/w) SDE, about 15 percent (w/w) dimethyl isosorbide, 14.1 percent (w/w) PEG 400, about 50 percent (w/w) diethylene glycol monoethyl ether, about 20 percent (w/w) diisopropyl adipate, and about 0.8 percent (w/w) hydroxypropylcellulose.

15. The pharmaceutical composition of claim 14, comprising about 4 percent (w/w) SDE.

16. The pharmaceutical composition of claim 1, comprising from about 0.1 to about 3.2 percent (w/w) SDE, from about 5 to about 25 percent (w/w) isopropyl myristate, from about 0 to about 10 percent (w/w) oleyl alcohol, from about 0 to about 20 percent (w/w) castor oil, about 5 to about 15 percent (w/w) Caprylic/Capric Triglyceride, about 0 to about 25 percent (w/w) liquid paraffin, about 10 to about 30 percent (w/w) diisopropyl adipate, from about 26 to about 45 percent (w/w) soft paraffin, and about 0 to 8 percent (w/w) stearic acid.

17. The pharmaceutical composition of claim 16, comprising from about 0.1 to about 2 percent (w/w) SDE, about 16 percent (w/w) isopropyl myristate, about 14 percent (w/w) castor oil, about 10 percent (w/w) Caprylic/Capric Triglyceride, about 20 percent (w/w) diisopropyl adipate, about 32 percent (w/w) soft paraffin, and about 0-8 percent (w/w) stearic acid.

18. The pharmaceutical composition of claim 16, comprising from about 0.1 to about 2 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, from about 32 to 37 percent (w/w) soft paraffin, and from about 0 to about 4 percent (w/w) stearic acid.

19. The pharmaceutical composition of claim 16, comprising from about 1 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, about 33 percent (w/w) soft paraffin, and about 4 percent (w/w) stearic acid.

20. The pharmaceutical composition of claim 16, comprising from about 1 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, and about 37 percent (w/w) soft paraffin.

21. The pharmaceutical composition of claim 19 or claim 20, further comprising an antioxidant.

22. The pharmaceutical composition of claim 1, further comprising a solvent and a penetration enhancer.

23. The pharmaceutical composition of claim 22, comprising from about 0.1 to about 1 percent (w/w) SDE, from about 79 to about 90 percent (w/w) solvent, and from about 9 to 20 percent (w/w) penetration enhancer.

24. The pharmaceutical composition of claim 23, comprising from about 0.1 to about 1 percent (w/w) SDE, from about 10 to about 25 percent (w/w) isopropyl myristate, from about 9 to about 20 percent (w/w) castor oil, from about 5 to about 15 percent (w/w) Caprylic/Capric Triglyceride, from about 14 to about 30 percent (w/w) diisopropyl adipate, and from about 26-45 percent (w/w) liquid paraffin.

25. The pharmaceutical composition of claim 14, comprising from about 1 percent (w/w) SDE, about 18 percent (w/w) isopropyl myristate, about 14 percent (w/w) castor oil, about 10 percent (w/w) Caprylic/Capric Triglyceride, about 20 percent (w/w) diisopropyl adipate, and from about 37 percent (w/w) liquid paraffin.

26. The pharmaceutical composition of claim 25, further comprising an antioxidant.

27. A method of treating disorders, comprising topically administering a pharmaceutically effective amount of a non-aqueous pharmaceutical composition comprising SDE or a pharmaceutically acceptable salt thereof and at least one pharmaceutically acceptable excipient to a subject in need thereof.

28. The method of claim 27, wherein the disorders are selected from pruritus conditions, pain, or inflammatory conditions.

29. The method of claim 28, wherein the disorder is pruritus condition.

30. The method of claim 29, wherein the pruritus condition is uremic pruritus, atopic dermatitis, or prurigo nodularis.

31. The method of claim 27, wherein the pharmaceutical composition is administered three times a day, twice a day, once a day, or every 2, 3, 4, 5, 6, or 7 days.

32. The method of claim 27, wherein the pharmaceutical composition releases nalbuphine from SDE over the course of 2, 4, 6, 8, 12, 24, 48, or 72 hours.

33. The method of claim 27, wherein the pharmaceutical composition is administered as a topical gel, topical ointment, topical lotion, topical foam, or topical cream.

34. The method of claim 27, wherein the SDE concentration in the dermis and epidermis at 24 hours post administration is higher than the SDE concentration in the circulation.

35. The method of claim 29, wherein the symptoms of pruritus are alleviated or partially alleviated.

36. The method of claim 27, wherein the subject does not experience opioid-related side effects.
